(19) United States
(12) Patent Application Publication
(10) Pub. No.: US 2024/0342233 A1
(43) Pub. Date: Oct. 17, 2024

(54) Herbal composition for prostate health and prostate cancer prevention

(71) Applicant: Laura Mann Kevehazi, Hertfordshire (GB)

(72) Inventor: Laura Mann Kevehazi, Hertfordshire (GB)

(21) Appl. No.: 18/749,877

(22) Filed: Jun. 21, 2024

Related U.S. Application Data
(63) Continuation of application No. PCT/EP2023/054192, filed on Feb. 20, 2023.
(60) Provisional application No. 63/313,018, filed on Feb. 23, 2022.

(51) Int. Cl. A61K 36/42; A61K 31/01; A61K 31/4375; A61K 33/04; A61K 36/074; A61K 36/185; A61P 13/08

(52) U.S. Cl. CPC A61K 36/42; A61K 31/01; A61K 31/4375; A61K 33/04; A61K 36/074; A61K 36/185; A61P 13/08

(57) ABSTRACT

A composition for protecting the prostate tissue and inhibiting prostate cancer development has Pumpkin seed extract; Nettle root extract; Berberine; Embilica officinalis (Amla berry); Ganoderma Lucidum (Reishi mushroom); Lycopene; and Selenium.

### CROSS REFERENCE TO RELATED APPLICATIONS

In accordance with 37 CFR 1.76, a claim of priority is included in an Application Data Sheet filed concurrently herewith. Accordingly, the present invention claims priority as a continuation application to International (PCT) Patent Application No. PCT/EP2023/054192, entitled “HERBAL COMPOSITION FOR PROSTATE HEALTH AND PROSTATE CANCER PREVENTION”, filed Feb. 20, 2023, which claims the benefit of the priority of U.S. Provisional Patent Application No. 63/313,018, entitled “HERBAL COMPOSITION FOR PROSTATE HEALTH AND PROSTATE CANCER PREVENTION” filed Feb. 23, 2022, the contents of each are incorporated herein by reference.

### FIELD OF THE INVENTION

The present invention relates to compositions which can be used to protect the prostate tissue and inhibit prostate cancer development, as well as mitigating urinary tract symptoms caused by prostate enlargement. More particularly, the composition comprises multiple botanical/herbal extracts and mineral ingredients protect the prostate tissue and inhibit prostate cancer development.

### BACKGROUND OF THE INVENTION

The prostate gland is intimately involved in the health of any man. With age Benign Prostate Hyperplasia (BPH) becomes the most common problem impacting men's lives for decades. According to the American Urology Association, half of men between ages 51-60 have documented Benign Prostate Hyperplasia (BPH) and close to 90% suffer from Lower Urinary Tract Symptoms (LUTS).

Prostate enlargement is a long term condition fueled by chronic inflammation at cellular level. The link between chronic inflammation and carcinogenesis was proven in multiple studies. Research at John Hopkins School of Medicine has shown that in spite of low PSA levels, chronic inflammation in the prostate more than doubles the risk of aggressive prostate cancer. Prostate cancer affects one in six Caucasian men and a staggering one in four men of African ethnic background.

Fifty years ago, President Richard Nixon declared ‘war on cancer’ and signed it into law hoping to find a cure. Yet, in spite of the huge amounts of funding and research invested in finding a cure, fifty years down the line, in spite of extensive national screening, cancer continues to be a life altering diagnostic affecting 1 in 6 or 1 in 4 men and their families. The current screening and diagnostic procedures for prostate cancer are invasive scans (Transrectal Ultrasound, Transrectal Prostate Biopsy) with a high risk of infection, can lead to a high amount of false negatives or false positives, as well as being expensive, and very unpleasant for the patient.

The cancer treatments include transurethral Prostatectomy, radiation, and chemotherapy with a high risk for infection and serious side effects: impotence, urethral scarring, urinary incontinence, chronic urinary tract infections, urinary retention that require catheterization, and sepsis by way of non limiting example. These are a heavy burden on the healthcare system and a huge toll on the patients mental health and their families. Currently, there are no safe and effective ways to prevent prostate carcinogenesis at cellular level before a tumor is formed.

There is an urgent need to reduce the high rates of prostate malignancy affecting one in six Caucasian men and one in four men of African ethnicity. At the same time there is a need for a safe and affordable solution for mitigating the urinary symptoms caused by Benign Prostate Hyperplasia that affect the overwhelming majority of men over the age of 50.

The present invention has been devised in light of the above considerations.

### BRIEF DESCRIPTION OF THE DRAWINGS

The present disclosure will be better understood by reading the written description with reference to the accompanying drawing figures in which like references numerals denote similar structure and referred to like elements throughout which:

FIG. 1 is a graphical representation of the efficacy of the formula used in accordance with the invention.

FIG. 2 is field images of PC-3 cells, before application of current tested composition (non-treated) as well as 24 and 48 hours after the application.

FIG. 3 is a graphical representation of the efficacy of the composition-specifically the inhibitory effect on the viability of the Prostate cancer cells used in accordance with the invention, as well as the synergistic effect of the composition compared to the individual ingredients (Pumpkin seed extract, Nettle root, Reishi mushroom, Amla berry and Berberine alone) and a control.

### SUMMARY OF THE INVENTION

A first aspect of the present invention provides a composition comprising Pumpkin seed extract; Nettle root extract (Urtica doica); Berberine; Embilica officinalis (Amla berry); Ganoderma Lucidum (Reishi mushroom); Lycopene; and Selenium.

More particularly in one embodiment the composition is in the form of tablets or capsules and comprises Pumpkin seed extract to from 500-1000 mg per daily dose; Nettle root extract (Urtica doica) to between 300-700 mg per daily dose; Berberine Hydrochloride 90% from 50-100 mg per daily dose; Embilica officinalis (Amla berry) from 50-100 mg per daily dose; Ganoderma Lucidum (Reishi mushroom) from 50-100 mg per daily dose; Lycopene from 5-10 mg per daily dose; and Selenium in the form of L-Selenomethionine, Sodium selenite or Sodium selenate from 30-40 mcg per daily dose. The recommended daily dose amount can be reached by a single dosage form such as a liquid or gel, or by several tablets or capsules.

In another embodiment of the present invention the composition comprises: Pumpkin seed extract equivalent to between 1000-4000 mg Pumpkin seed per dose; Nettle root extract (Urtica doica) equivalent to between 500-2000 mg Nettle root per dose; Berberine Hydrochloride 90% 50-300 mg per dose; Embilica officinalis (Amla berry) 50-300 mg per dose; Ganoderma Lucidum (Reishi mushroom) 50-300 mg per dose; Lycopene 5-20 mg per dose; and Selenium from 40-160 mcg per dose. In a further aspect the composition is in the form of a powder. In another aspect the composition is in the form of a liquid or a gel.

In another aspect of the invention the composition is in the form of a liquid, gel or powder comprising: 400 mg of Pumpkin seed extract 10:1 equivalent of 4000 mg Pumpkin seed; 200 mg of Nettle root extract 10:1 (Urtica doica) equivalent to 2000 mg Nettle root; 200-300 mg of Berberine Hydrochloride; 200-300 mg of Embilica Officinalis (Amla berry); 200-300 mg of Ganoderma lucidum (Reishi mushroom); 20 mg of Lycopene; and 160 mcg of Selenium. In some embodiments the liquid, gel or powder is administered as a single daily dose.

In another embodiment of the present invention the composition is in the form of a tablet or a capsule comprising: Pumpkin seed extract equivalent to 1000-2000 mg Pumpkin seed; Nettle root extract (Urtica doica) equivalent to 500-1000 mg Nettle root; 50-150 mg of Berberine Hydrochloride 90%; 50-150 mg of Embilica officinalis (Amla berry); 50-150 mg of Ganoderma Lucidum (Reishi mushroom); 5-10 mg of Lycopene; and 40-80 mcg of Selenium. In further embodiments the composition is in the form of a tablet or a capsule comprising: Pumpkin seed extract equivalent to 1000 mg of Pumpkin seed; Nettle root extract (Urtica doica) equivalent to 500 mg of Nettle root; 50 mg of Berberine Hydrochloride 90%; 50 mg of Embilica officinalis (Amla berry); 50 mg of Ganoderma Lucidum (Reishi mushroom) per dose; 5 mg of Lycopene; and 40 mcg of Selenium. In some embodiments 2-4 tablets/capsules can be administered per day.

In yet another embodiment of the present invention the composition is in the form of a liquid or gel having Pumpkin seed extract equivalent to about 4000 mg Pumpkin seed per daily dose; Nettle root extract (Urtica doica) equivalent to about 2000 mg Nettle root per daily dose; Berberine Hydrochloride 90% of about 200 mg per daily dose; Embilica officinalis (Amla berry) of about 200 mg per daily dose; Ganoderma Lucidum (Reishi mushroom) of about 200 mg per daily dose; Lycopene 15 of about 20 mg per daily dose; and Selenium of about 160 mcg per daily dose.

In still another embodiment of the present invention the composition is in the form of a tablet having Pumpkin seed extract between 1000-4000 mg per daily dose; Nettle root extract (Urtica doica) between 500-2000 mg per daily dose; Berberine Hydrochloride 90% between 50-200 mg per daily dose; Embilica officinalis (Amla berry) between 50-200 mg per daily dose; Ganoderma Lucidum (Reishi mushroom) between 50-200 mg per daily dose; Lycopene between 5-20 mg per daily dose; and Selenium from 4-160 mcg per daily dose. In some embodiments 2-4 tablets can be administered per day.

In some embodiments, the compositions of the present invention comprise pharmaceutically acceptable excipients. Excipients for different embodiments, capsules, soft capsules, gel, powder sachet or liquid may include, as illustrative examples, one or more of: gelling agent(s), surfactant(s), emulsifier(s), solubilizer(s), surfactant(s) (nonionic), plasticizer(s), matrix forming agent(s), sweetener(s), bulking/diluent agent(s), preservative(s), anti-caking/lubricant(s), coating agent(s). Illustrative embodiments such excipients may include, PEG-35 Castor Oil, Polysorbate 80, Poloxamers, Polyethyleneglycol-15-hydroxystearate, Stevia (sweetener), microcrystalline cellulose (bulking/diluent agents), or silicon dioxide (anti caking/lubricant agents). The above listed compounds are illustrative only. Other excipients known to one of skill in the art may be used in addition to, or alternative to. In further embodiments, the composition may comprise one or more of pharmaceutically acceptable diluent(s), lubricant(s), glazing and coating agent(s). In another aspect of the invention the compositions are for preventing prostate cancer, protecting the prostate cells from malignant changes and significantly lowering the risk of prostate cancer.

In another embodiment the compositions are for use in mitigating the urinary symptoms of an enlarged prostate. In a further embodiment the compositions are for use in treating urinary symptoms triggered by an enlarged prostate optionally wherein the urinary symptoms are selected from frequency, nocturia, straining and urgency of urination. In another embodiment the compositions are for use in treating erectile dysfunction or improving sexual function.

Also disclosed is a method of preventing prostate cancer and protecting the prostate cells from malignant changes may comprise, administering to an individual in need thereof, any composition (in any concentrations or dosages) as described or contemplated herein (i.e., comprising Pumpkin seed extract; Nettle root extract (Urtica doica); Berberine Hydrochloride; Embilica officinalis (Amla berry); Ganoderma Lucidum (Reishi mushroom); Lycopene; and Selenium; and optionally one or more of pharmaceutically acceptable excipients or diluents, lubricants, glazing and coating agents.

Also disclosed is a method of lowering or reducing the risk of prostate cancer and protecting the prostate cells from malignant changes may comprise, administering to an individual in need thereof, any composition (in any concentrations or dosages) as described or contemplated herein (i.e., comprising Pumpkin seed extract; Nettle root extract (Urtica doica); Berberine Hydrochloride; Embilica officinalis (Amla berry); Ganoderma Lucidum (Reishi mushroom); Lycopene; and Selenium; and optionally one or more of pharmaceutically acceptable excipients or diluents, lubricants, glazing and coating agents.

Also disclosed is a method of mitigating the urinary symptoms of an enlarged prostate may comprise, administering to an individual in need thereof, any composition (in any concentrations or dosages) as described or contemplated herein (i.e., comprising Pumpkin seed extract; Nettle root extract (Urtica doica); Berberine Hydrochloride; Embilica officinalis (Amla berry); Ganoderma Lucidum (Reishi mushroom); Lycopene; and Selenium; and optionally one or more of pharmaceutically acceptable excipients or diluents, lubricants, glazing and coating agents.

Also disclosed is a method of treating urinary symptoms triggered by an enlarged prostate may comprise, administering to an individual in need thereof, any composition (in any concentrations or dosages) as described or contemplated herein (i.e., comprising Pumpkin seed extract; Nettle root extract (Urtica doica); Berberine Hydrochloride; Embilica officinalis (Amla berry); Ganoderma Lucidum (Reishi mushroom); Lycopene; and Selenium; and optionally one or more of pharmaceutically acceptable excipients or diluents, lubricants, glazing and coating agents.

Also disclosed is a method of treating urinary symptoms triggered by an enlarged prostate, optionally wherein the urinary symptoms are selected from frequency, nocturia, straining and urgency of urination, may comprise, administering to an individual in need thereof, any composition (in any concentrations or dosages) as described or contemplated herein (i.e., comprising Pumpkin seed extract; Nettle root extract (Urtica doica); Berberine Hydrochloride; Embilica officinalis (Amla berry); Ganoderma Lucidum (Reishi mushroom); Lycopene; and Selenium; and optionally one or more of pharmaceutically acceptable excipients, or diluents, lubricants, glazing and coating agents.

The invention includes the combination of the aspects and preferred features described except where such a combination is clearly impermissible or expressly avoided.

### DETAILED DESCRIPTION OF THE PREFERRED EMBODIMENTS

Aspects and embodiments of the present invention will now be discussed. Further aspects and embodiments will be apparent to those skilled in the art.

Modern lifestyles involve exposure to a variety of carcinogenic factors: smoking, chemicals, outdoor or indoor pollution, radiation or toxins which generate tiny particles called free radicals. Free radicals can damage all major components of cells, including DNA, proteins, and cell membranes leading to oxidative stress, chronic inflammation and cellular disfunction setting up the process of carcinogenesis.

Cancer is a complex disease driven by chronic inflammation through metabolic pathways, proven to actively contribute to cancer initiation, promotion and progression. Prostate cancer as used herein is a generic term for tumors of the prostate as each tumor consists of genetically different diverse cancer cells and targeting just one or two processes leads to rapid additional mutations evading the targeted therapies.

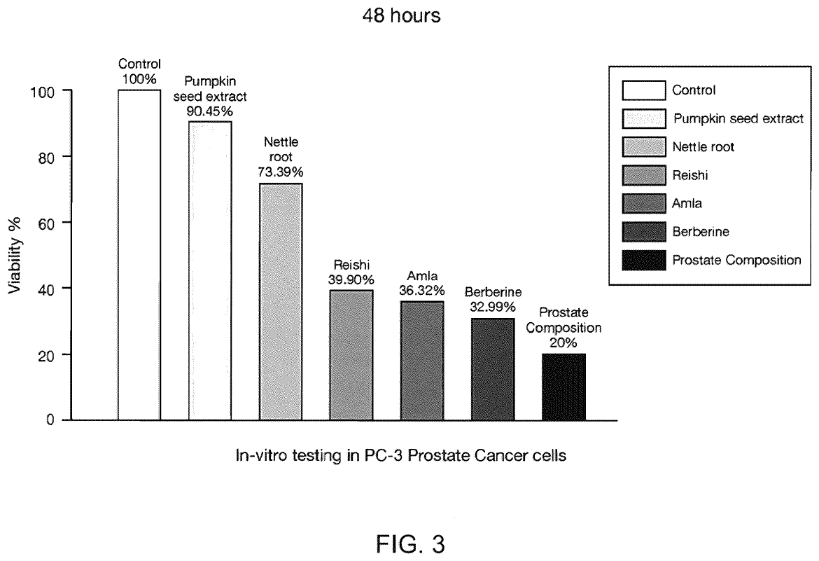

As a result, no single solution or approach to date provides sustainable result providing both cellular protection and improved bladder and urinary comfort. Using just one ingredient can have side effects and will not comprehensively address all the mechanisms through which cancer takes hold in our cells, develops and spreads. The above ingredients synergistically provide the desired benefits; more than each one individually. This synergistic effect of the composition compared to separate ingredients is demonstrated by Example 1 and FIG. 3.

This is the precise reason why the present composition includes several ingredients, each addressing several aspects of cancer cell metabolism: antioxidant activity, anti-inflammatory activity, modulation of inflammatory enzymes, immune response, inhibition of rapid multiplication by inducing apoptosis, inhibition of angiogenesis, inhibition of enzymes that enable spread into the extracellular matrix, and activation of detoxifying mechanisms. The combination of the various phytochemicals and minerals in lower doses avoids the possible side effects of several ingredients providing synergistic effects with each other while avoiding toxicity. The potential exists for the current herbal and mineral composition to provide cellular antioxidant protection, as well as prophylactic and potentially chemo preventive effect with daily administration.

Daily amounts of phytochemicals, antioxidants and minerals as described below, targeted for cellular prostate health, effectively neutralize free radicals, mitigate chronic inflammatory pathways, prevent and combat cellular damage, representing a natural, nontoxic, and affordable long-term chemoprevention. In this composition low amounts of each ingredient are included in order to provide a desired chemo-preventive fighting effect while also mitigating the chronic inflammation present in BPH and its effects on the bladder function while avoiding any possible side effects.

The proposed formulations include the following ingredients:

EMBILICA OFFICINALIS (Amla Berry) at 260,000 ORAC units, the amla berry has the highest antioxidant capacity (free radical scavenging capacity) of all fruits and vegetables; that is 27 times more than wild blueberries at 9,600 ORAC units. Amla's high level of antioxidant capacity may be due to high content of content of vitamin C, polyphenols, such as flavonoids and hydrolysable tannins, that exert its strong anti-inflammatory activity, modulation of various enzymes of inflammation like NF-kB which is one of the main drivers of carcinogenesis leading to apoptosis in neoplastic cells. The amount of Embilica officinalis in the composition is in the range of 50-300 mg per dose. In further embodiments the amount of Embilica officinalis in the composition is in the range of 50-200 mg per dose. The daily dose of Embilica officinalis is 200-300 mg.

GANODERMA LUCIDUM (Reishi mushroom) contains beta-glucan polysaccharides and triterpenoids. The chemo preventive activity of Reishi is exerted through multiple mechanisms: downregulation of estrogen-alpha receptors and NF-kB pathway, antiangiogenic effect through down regulation of VGEF, inhibition of metalloproteinase (MMP-9) downregulates cellular migration and induction of apoptosis in cancer cells. Prostate tissue has an abundance of Estrogen receptors, both ER-alpha and ER-beta that drive carcinogenesis, and upregulate tumor development. Ganoderma down regulates the Estrogen-alpha receptors found to mediate high grade prostatic intraepithelial neoplasia. The Ganoderma Lucidum can be in the form of the fruiting body of the mushroom, which is the stalk and cap, and comprises the organism's reproductive structure. The amount of Ganoderma in the composition is in the range of 50-300 mg per dose. In further embodiments the amount of Ganoderma lucidum in the composition is in the range of 50-200 mg per dose. The daily dose of Ganoderma lucidum is 200-300 mg.

BERBERINE—Berberine is an isoquinoline plant alkaloid with the chemical formula C₂₀H₁₈CINO₄, widely present in different traditional medicines. In the present composition an orally available synthetic form of Berberine was used which is Berberine Hydrochloride with the chemical formula C₂₀H₂₂CINO₆. It was used in the present composition for its direct effect on prostatic stromal and glandular components, known to be involved in the development of BPH. Berberine acts on the prostate cells through inhibiting the inflammatory microenvironment at cellular level, interfering with the metabolic changes leading to cancer initiation, inhibiting carcinogenesis and inducing prostate cancer cells apoptosis. Berberine in high amounts can cause altered liver function, gastric symptoms, hepato and hematotoxicity, hemorrhagic inflammatory consequences, damage to immune cells. Therefore the amount of Berberine in the composition is in the range of 50-300 mg per dose. In further embodiments the amount of Berberine in the composition is in the range of 50-200 mg per dose. The daily dose of Berberine is 200-300 mg. In some embodiments the Berberine is in the synthetic form Berberine Hydrochloride (C₂₀H₂₂CINO₆).

PUMPKIN SEED EXTRACT—has an antioxidant and anti-inflammatory effect while having a dual beneficial effect on the bladder and prostate. It directly contributes to improvement in the urinary symptoms caused by BPH: frequency, urgency, nocturia, weak stream and straining, thus greatly improving the quality of life of men affected by BPH. Pumpkin seed extract is used in amounts of 500-4000 mg per daily dose. The pumpkin seed extract can be water soluble. The Pumpkin seed extract used can be Pumpkin seed extract 10:1. Pumpkin seed extract 10:1 (1 mg extract is the equivalent to 10 mg of pumpkin seed) means the daily dose equivalent to 4000 mg is reached by 400 mg of extract. In further embodiments the amount of pumpkin seed extract in the composition is in an amount equivalent to 1000-4000 mg Pumpkin seed per dose. The daily dose of Pumpkin seed extract is equivalent to 4000 mg Pumpkin seed.

NETTLE ROOT EXTRACT—(Urtica doica) contains multiple antioxidant and anti-inflammatory molecules (quercetin, rutin, kampfaerol) and it is used in the current composition for its effects on mitigating prostate enlargement; Benign Prostate Hyperplasia (BPH). Stinging nettle blocks the oxidation of fats thereby protecting many tissues from oxidative stress, as well as reducing inflammatory cytokine release and inflammatory biomarkers like TF-kB and interleukins, thus exerting an antiproliferative effect on prostate cells. Nettle root extract increases the effect of blood pressure medication as well as diabetes medication, hence using it in the correct amounts can simultaneously provide a chemopreventive effect against other medications as well as mitigate the effects of an enlarged prostate, have a beneficial effect on the bladder that is inevitably negatively impacted by an enlarged prostate. The Nettle root extract used can be Nettle root extract 10:1. For example Nettle root extract 10:1 (1 mg extract is the equivalent to 10 mg of Nettle root) means the daily dose equivalent to 2000 mg is reached by 200 mg of extract. Nettle root extract is used in amounts 500-2000 mg per dose. The daily dose of Nettle root extract is equivalent to 2000 mg Nettle root.

LYCOPENE—is the red pigment found in tomatoes, and the most powerful antioxidant in the carotenoid family. It is an oxidant, thus protecting the prostate cells from oxidative stress which can cause chronic inflammation. It plays an important role in cardiovascular health supporting a healthy blood flow and improving capillary circulation. It has a dual effect of protecting the prostate cells from oxidative damage while its improved capillary blood flow effect enables and increases the effects of all the ingredients in the current composition through enhanced delivery at cellular level. As Lycopene may increase the risk of bleeding in people taking anti-coagulants, its use should be in adequate amounts. Lycopene is used in in the current composition in a range of 5-20 mg per dose. The daily dose of Lycopene is 20 mg.

SELENIUM is a micronutrient that humans cannot produce and need in daily amounts for health maintenance. In the current composition Selenium has a protective effect at cellular level and cancer prevention potential. It exerts its protective actions through multiple mechanisms that involve antioxidant activities protecting the prostate cells from oxidative damage and inhibition of DNA damage, inducing apoptosis, inhibiting angiogenesis and invasion. There is a known connection between selenium supplementation and chemo-protective anti-cancer activity. This biological phenomenon may be due to the ability of selenium to instigate cellular apoptosis. However, Selenium is toxic at doses over 400 mcg, causing peripheral neuropathy, gastric symptoms, liver, kidney and heart problems, and alopecia. Due to its toxicity, selenium cannot be used as a single ingredient (a dose of 750 mg-represents 750,000 mcg-would be lethal) it can only be used in very small amounts, and in order to benefit from its protective effects against carcinogenesis is used in the current formulation in very small amounts for the synergistic effect it delivers in combination with the other ingredients.

Selenium is used in the range of 40-160 mcg per dose. The daily dose of selenium is 160 mg. In some embodiments the Selenium in the form of one of L-Selenomethionine, Sodium selenate (Na₂SeO₄) or Sodium selenite (Na₂SeO₃). In further embodiments the Selenium is in the form of Sodium selenate or Sodium selenite.

Generally the inventive composition includes Pumpkin seed extract, Nettle root extract (Urtica doica); Berberine; Embilica officinalis (Amla berry); Ganoderma Lucidum (Reishi mushroom); Lycopene; and Selenium.

More particularly in a first embodiment each dose of the composition includes equivalent to 1000-4000 mg of Pumpkin seed (extract); equivalent to 500-2000 mg of Nettle root (extract) (Urtica doica); 50-200 mg of Berberine Hydrochloride 90%; 50-200 mg of Embilica officinalis (Amla berry); 50-200 mg of Ganoderma Lucidum (Reishi mushroom); 5-20 mg of Lycopene; and 40-160 mcg of one of Selenium in the form of L-Selenomethionine or Sodium selenate.

In another embodiment of the present invention the composition is in the form of a powder. Each dose of the composition includes 1000-4000 mg of Pumpkin seed extract; 500-2000 mg of Nettle root extract (Urtica doica); 50-200 mg of Berberine Hydrochloride 90%; 50-200 mg of Embilica officinalis (Amla berry); 50-200 mg of Ganoderma Lucidum (Reishi mushroom); 5-20 mg of Lycopene; and 40-160 mcg of Selenium.

In yet another embodiment of the present invention the composition is in the form of a liquid or gel, each dose having the equivalent of between 1000-4000 mg Pumpkin seed extract; the equivalent of between 500-2000 mg of Nettle root extract (Urtica doica); 50-200 mg of Berberine Hydrochloride 90%; 50-200 mg of Embilica officinalis (Amla berry); 50-200 mg of Ganoderma Lucidum (Reishi mushroom); 5-20 mg Lycopene; and 40-160 mcg Selenium. In still another embodiment of the present invention the composition is in the form of a tablet Each tablet of the composition includes 1000-4000 mg of Pumpkin seed extract; 500-2000 mg of Nettle root extract (Urtica doica); 50-200 mg of Berberine Hydrochloride 90%; 50-200 mg of Embilica officinalis (Amla berry); 50-200 mg of Ganoderma Lucidum (Reishi mushroom); 5-20 mg of Lycopene; and 40-160 mcg of Selenium.

In a preferred embodiment a capsule or tablet format comprises per tablet/capsule dose:
Pumpkin seed extract equivalent to: 1000 mg
Nettle root extract (Urtica doica) equivalent to: 50 mg
Berberine Hydrochloride 90%: 50 mg
Embilica Officinalis (Amla berry): 50 mg
Ganoderma Lucidum (Reishi mushroom): 50 mg
Lycopene: mg
Selenium: 40 mcg.
2-4 capsules/tablets can be administered per day to achieve the preferred daily dose.

The preferred daily dose of the present composition is:
Pumpkin seed extract equivalent to 4000 mg of Pumpkin seed;
Nettle root extract (Urtica doica) equivalent to 2000 mg of Nettle root;
Berberine Hydrochloride 200-300 mg;
Embilica Officinalis (Amla berry) 200-300 mg;
Ganoderma Lucidum (Reishi mushroom) 200-300 mg;
Lycopene 20 mg; and
Selenium 160 mcg.

The preferred daily dose can be provided as a single dose in the form of a gel, a powder or a liquid. Or when the preferred daily dose is provided in capsule or tablet format this can be achieved by 2-4 capsules or tablets depending on their size. The compositions in the form of liquid or gel can be in a suitable vehicle such as Polyethylene glycol (“PEG”) or coconut oil with added flavourings and sweeteners like stevia. The compositions described herein may consist of the above listed ingredients and optionally also including pharmaceutically acceptable excipient, bulking agents, in the form of a vegetarian capsule for example a Hydroxypropyl Methylcellulose capsule. The compositions described herein may consist of the above listed ingredients in the form of a tablet, and optionally also including pharmaceutically acceptable excipient, bulking agents, glazing agents and coating agents.

A further aspect of the present invention is the composition for use in combating and reliving the symptoms of prostate enlargement, the composition for use as prevention of prostate cancer. The compositions of the present invention may be used daily. As a result of the synergies amongst the ingredients, the proposed composition taken daily acts through different mechanisms on multiple cellular pathways, having an inhibitory effect on prostate tumor initiation and development as demonstrated by laboratory experiment described above (See Example 1). Due to the way ingredients work synergistically, the current composition was also shown to improve the urinary symptoms triggered by an enlarged prostate as demonstrated by the pilot study shown above (See Example 2). The unexpected effect observed after 6 months of use is an improvement in sexual function, both in terms of erectile function as well as frequency of sexual activity, as reported by all the subjects.

### Administration Protocol

Preferred administration is 2-4 capsules/tablets depending on the tablet/capsule size, or one gel sachet daily taken with food.
DOSE—the composition could take the form of a powder in a capsule wherein the capsule is a vegetarian capsule of Hydroxypropyl Methylcellulose. The composition can also be administered in the form tablet, liquid or gel in a sachet. The gel embodiment can include Phosphatidylcholine, flavorings, natural sweeteners like for example, stevia and preservatives. The capsule and tablet embodiments may also include pharmaceutically acceptable flowing agents, anti-caking agents and coating agents.

### EXAMPLES

### Example 1

Objective of the experiment was to examine the effect of the current composition on prostate cancer cells PC-3 cell line viability and apoptosis.

### Materials

PC-3 Prostate Cancer Cells (human prostate adenocarcinoma derived cell line ATCC CRL-1435) were used. To prepare growth medium for PC-3 cell line, Nutrient Mixture F-12 (Ham's) with L-glutamine was supplemented with 10% v/v FBS, 1% v/v sodium pyruvate and 1% v/v Penicillin-Streptomycin. The medium was stored at 4 degrees Celsius until use. The composition was dissolved in Dulbecco's phosphate-buffered saline (“DPBS”).

### Procedure

PC-3 cell lines were cultured in T75 flasks in full growth medium for 3 passages at 370 C in 5% CO2. 10,000 cells/well were seeded in two 96-well plates.

### Methods

Plates were incubated overnight at 37° C. in 5% CO2 to allow attachment. Fresh growth medium containing 10 mg/ml of the composition on was applied on PC-3 cells. One plate was incubated for 24 hours and the other plate was incubated for 48 hours at 370 C in 5% CO2 All the treatments were done in quadruplicate.

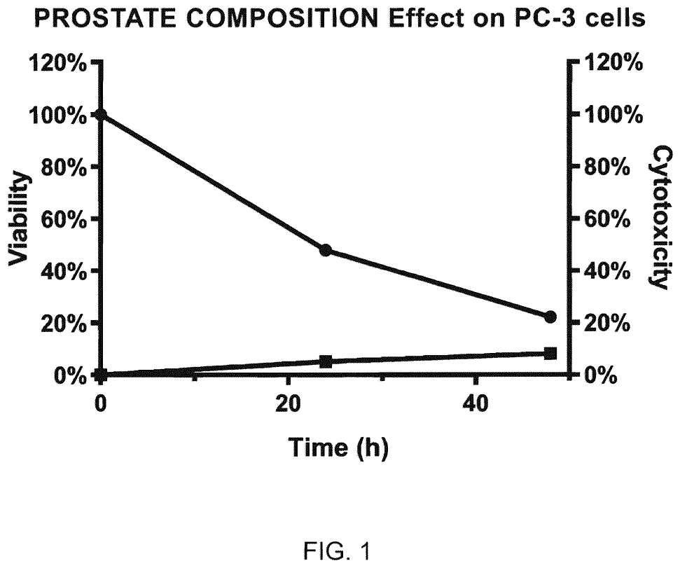

Cells used as control were treated with medium containing 5% DPBS as vehicle control. Results: Reference is made to FIG. 1 showing the effect of the composition on cell viability and cytotoxicity (apoptosis) following 24 and 48 hours after application of the current composition.

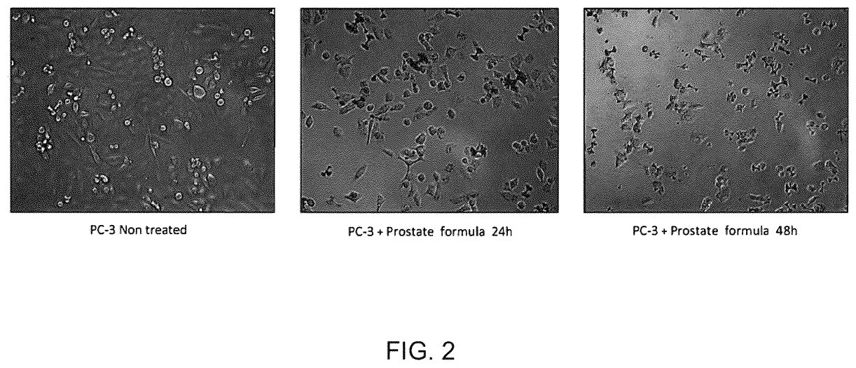

References is also made to field images of PC-3 cells in FIG. 2, before application of current tested composition (non treated) as well as 24 and 48 hours after the application.

Reference is made to FIG. 3 which shows the effect of the individual ingredients: pumpkin seed extract, Nettle root (Urtica doica), Reishi, Amla berry and Berberine; a control and the current composition on cell viability and cytotoxicity (apoptosis) following 48 hours after application. FIG. 3 demonstrates the synergistic anti-cancer effect of the current composition in comparison to the ingredients alone.

### Example 2

The current composition was also shown to improve the urinary symptoms triggered by an enlarged prostate as demonstrated by the pilot study shown below. This pilot study was done using the International Prostate Symptom Score (IPSS) developed by the American Urology Association and adopted as global standard. IPSS is the official worldwide assessment for prostate enlargement (BPH) based on answers to seven questions concerning urinary symptoms and one question concerning quality of life. Five male subjects aged 60-66 completed the questionnaire at the start of the study and after 3 months of daily administration of the proposed prostate health composition. Initial scoring shown below:
Subject 1 Moderate Score 16
Subject 2 Moderate Score 19
Subject 3 Moderate Score 18
Subject 4 Severe Score 28
Subject 5 Severe Score 31

The subjects started taking a daily dose of the Prostate health composition, taken with food. After 3 months they were asked to redo the questionnaire. All subjects indicated a significant improvement in urinary symptoms and comfort, for example:
Frequency—was improved by an average of 40%.
Initial average score was 4. After 3 months the average score was 2.4.
Nocturia—number of night bathroom runs, was improved by an average of 40%.
Initial average score 4.6. After 3 months the average score was 2.4.
Straining—was improved by an average of 25%.
Initial average score was 4. After 3 months the average score was 3.
Urgency—was improved by an average of 30%
Initial average score 3.4. After 3 months the average score was 2.4.
Quality of life due to urinary symptoms score improved from an average of 4 to an average of 2 representing 50% improvement.

Example 2 demonstrates that the present composition improves the urinary symptoms triggered by an enlarged prostate and the quality of life of the subject.

A follow-up of subjects after 6 months highlighted an unexpected effect of the prostate composition. All the subjects reported an improvement in sexual activity. This includes both a significant improvement in erectile function, that previously required medication, as well as duration and frequency of sexual activity which increased compared to the period before starting.

What is claimed is:

1. A composition comprising:
Pumpkin seed extract;
Nettle root extract (Urtica doica);
Berberine;
Embilica officinalis (Amla berry);
Ganoderma Lucidum (Reishi mushroom);
Lycopene; and
Selenium.

2. The composition according to claim 1 wherein the composition comprises:
the Pumpkin seed extract is in an amount equivalent to 1000-4000 mg Pumpkin seed per dose;
the Nettle root extract (Urtica doica) is in an amount equivalent to 500-2000 mg Nettle root per dose;
the Berberine in the form of Berberine Hydrochloride 90% is in an amount of 50-300 mg per dose;
the Embilica officinalis (Amla berry) is in an amount of 50-300 mg per dose;
the Ganoderma Lucidum (Reishi mushroom) is in an amount of 50-300 mg per dose;
the Lycopene is in an amount of 5-20 mg per dose; and
the Selenium is in an amount of 40-160 mcg.

3. The composition according to claim 1 wherein the composition comprises:
Pumpkin seed extract equivalent to 4000 mg of Pumpkin seed per daily dose;
Nettle root extract (Urtica doica) equivalent to 2000 mg of Nettle root per daily dose;
Berberine in the form of Berberine Hydrochloride 200-300 mg per daily dose;
Embilica Officinalis (Amla berry) 200-300 mg per daily dose;
Ganoderma Lucidum (Reishi mushroom) 200-300 mg per daily dose;
Lycopene 20 mg per daily dose; and
Selenium 160 mcg per daily dose.

4. The composition according to claim 1, wherein the composition is in the form of one of a powder, liquid or a gel.

5. The composition of claim 1, wherein the composition is in the form of a liquid, gel or powder comprising:
400 mg of 10:1 Pumpkin seed extract equivalent to 4000 mg Pumpkin seed;
200 mg of 10:1 Nettle root (Urtica doica) extract equivalent to 2000 mg Nettle root;
200-300 mg of Berberine Hydrochloride;
200-300 mg of Embilica Officinalis (Amla berry);
200-300 mg of Ganoderma lucidum (Reishi mushroom);
20 mg of Lycopene; and
160 mcg of Selenium.

6. The composition of claim 1, wherein the composition is in the form of a tablet or capsule comprising:
Pumpkin seed extract equivalent to 1000-2000 mg Pumpkin seed;
Nettle root extract (Urtica doica) equivalent to 500-1000 mg Nettle root;
50-150 mg of Berberine Hydrochloride 90%;
50-150 mg of Embilica officinalis (Amla berry);
50-150 mg of Ganoderma Lucidum (Reishi mushroom);
5-10 mg of Lycopene; and
40-80 mcg of Selenium.

7. The composition according to claim 1, wherein composition is the form of a powder in a capsule optionally wherein the capsule is a vegetarian capsule of Hydroxypropyl Methylcellulose.

8. The composition of claim 1, further comprising one or more of pharmaceutically acceptable diluents, lubricants, glazing and coating agents.

9. The composition according to claim 1 wherein the Selenium is in the form of one of L-Selenomethionine, sodium selenate or sodium selenite.

10. A composition for use in preventing prostate cancer and protecting the prostate cells from malignant changes or mitigating the urinary symptoms of an enlarged prostate comprising:
Pumpkin seed extract;
Nettle root extract (Urtica doica);
Berberine Hydrochloride;
Embilica officinalis (Amla berry);
Ganoderma Lucidum (Reishi mushroom);
Lycopene; and
Selenium; and
optionally one or more of pharmaceutically acceptable diluents, lubricants, glazing and coating agents.

11. The composition according to claim 1, for use in preventing prostate cancer and protecting the prostate cells from malignant changes with daily administration wherein the dose comprises:
Pumpkin seed extract is in an amount of 4000 mg Pumpkin seed per daily dose;
Nettle root extract (Urtica doica) is in an amount of 2000 mg Nettle root per daily dose;
Berberine Hydrochloride 90% is in an amount of 200-300 mg per daily dose;
Embilica officinalis (Amla berry) is in an amount of 200-300 mg per daily dose;
Ganoderma Lucidum (Reishi mushroom) is in an amount of 200-300 mg per daily dose;
Lycopene is in an amount of 20 mg per daily dose; and
Selenium is in an amount of 160 mcg in the form of one of L-Selenomethionine or Sodium selenate per daily dose.

12. The composition according to claim 2 wherein the composition comprises:
Pumpkin seed extract equivalent to 4000 mg of Pumpkin seed per daily dose;
Nettle root extract (Urtica doica) equivalent to 2000 mg of Nettle root per daily dose;
Berberine in the form of Berberine Hydrochloride 200-300 mg per daily dose;
Embilica Officinalis (Amla berry) 200-300 mg per daily dose;
Ganoderma Lucidum (Reishi mushroom) 200-300 mg per daily dose;
Lycopene 20 mg per daily dose; and
Selenium 160 mcg per daily dose.

13. The composition of claim 2, wherein the composition is in the form of a tablet or capsule comprising:
Pumpkin seed extract equivalent to 1000-2000 mg Pumpkin seed;
Nettle root extract (Urtica doica) equivalent to 500-1000 mg Nettle root;
50-150 mg of Berberine Hydrochloride 90%;
50-150 mg of Embilica officinalis (Amla berry);
50-150 mg of Ganoderma Lucidum (Reishi mushroom);
5-10 mg of Lycopene; and
40-80 mcg of Selenium.

14. A composition for use in lowering the risk of prostate cancer and protecting the prostate cells from malignant changes, mitigating the urinary symptoms of an enlarged prostate, treating urinary symptoms triggered by an enlarged prostate optionally wherein the urinary symptoms are selected from frequency, nocturia, straining and urgency of urination, or treating erectile dysfunction or improving sexual function, comprising:
Pumpkin seed extract in an amount equivalent to 1000-4000 mg Pumpkin seed per dose;
Nettle root extract (Urtica doica) in an amount equivalent to 500-2000 mg Nettle root per dose;
Berberine in the form of Berberine Hydrochloride 90% in an amount of 50-300 mg per dose;
Embilica officinalis (Amla berry) in an amount of 50-300 mg per dose;
Ganoderma Lucidum (Reishi mushroom) in an amount of 50-300 mg per dose;
Lycopene in an amount of 5-20 mg per dose;
Selenium is in an amount of 40-160 mcg; and
optionally, one or more of pharmaceutically acceptable diluents, lubricants, glazing and coating agents.
